(19) United States
(12) Patent Application Publication
(10) Pub. No.: US 2022/0135939 A1
(43) Pub. Date: May 5, 2022

(54) METHODS FOR DETECTING DISEASES AND DISORDERS CHARACTERIZED BY ABERRANT RED BLOOD CELL AGGREGATION

(71) Applicant: Amerimmune LLC, McLean, VA (US)

(72) Inventors: Oral ALPAN, Fairfax, VA (US); Matthew PLASSMEYER, Fairfax, VA (US)

(73) Assignee: Amerimmune LLC, McLean, VA (US)

(21) Appl. No.: 17/383,404

(22) Filed: Jul. 22, 2021

Related U.S. Application Data
(60) Provisional application No. 63/055,291, filed on Jul. 22, 2020.

(51) Int. Cl. C12N 5/00; G01N 1/40

(52) U.S. Cl. CPC C12N 5/0087; G01N 2001/4083; G01N 1/4077

(57) ABSTRACT

This invention addresses accurately and rapidly diagnosing diseases, disorders, or conditions characterized by aberrant red blood cell aggregation, including infections caused by RNA viruses, particularly those caused by positive-sense, single-stranded RNA viruses, known to cause human disease. Examples of such viruses include various betacoronaviruses, including SARS-CoV, MERS-CoV, and SARS-CoV-2, that later of which causes COVID-19, a potentially fatal illness.

### RELATED APPLICATION(S)

This application claims the benefit of and priority to, commonly owned, co-pending U.S. provisional patent application No. 63/055,291 (docket number AME-0010-PV), filed on 22 Jul. 2020. Any aforementioned priority application is hereby incorporated by reference in its entirety for any and all purposes.

### BACKGROUND OF THE INVENTION

Coronavirus Disease, 2019 (COVID-19), was first noted near the end of 2019 in Wuhan, China, quickly spread to many countries around the world, and was subsequently designated a pandemic by the World Health Organization (WHO). COVID-19 poses critical challenges for global health, research, medicine, economies, and societies. Given its rapid method of spread, severity of disease, and delayed presentation of symptoms exacerbating continued person-to-person spread, reliable and rapid diagnostic testing is critical to reduced transmission and improving global health, economies, and societies.

Regrettably, more than 18 months after recognition of the devastating COVID-19 pandemic, testing options are limited to serological (antibody) and molecular (RT-PCR) testing, with a litany of continuous problems, including test availability, continuously changing information, shortage of reagents, testing efficacy, sensitivity, and specificity [1].

### SUMMARY OF THE INVENTION

The object of this invention is address shortcomings in accurately and rapidly diagnosing diseases, disorders, or conditions characterized by aberrant red blood cell aggregation, including infections caused by RNA viruses, particularly those caused by positive-sense, single-stranded RNA viruses, known to cause human disease. Examples of such viruses include various betacoronaviruses, including SARS-CoV, MERS-CoV, and SARS-CoV-2, that later of which causes COVID-19, a potentially fatal illness.

One aspect of the invention concerns methods for detecting aberrant red blood cell aggregation. Such methods involve determining whether a blood sample, for example, a peripheral blood sample, obtained from a subject, for example, a human subject, known or suspected to be afflicted with a disease or disorder characterized by aberrant red blood cell aggregation, contains pathologic red blood cell aggregation, and if so, indicating that aberrant red blood cell aggregation has been detected in the sample. Here, “aberrant” means abnormal or disease-associated and “pathologic” means involving, caused by, or being part of the nature of a disease or health disorder.

In some embodiments of this aspect, the disease or disorder characterized by aberrant red blood cell aggregation is selected from the group consisting of thrombosis, optionally an ischemic stroke; myocardial infarction; pulmonary embolism; deep vein thrombosis; and an infection, optionally a viral infection, optionally a viral infection caused by a positive-sense, single-stranded RNA virus, optionally a betacoronavirus, optionally SARS-CoV-2, SARS-CoV, or MERS-CoV

In some embodiments of this aspect, the presence of aberrant red blood cell aggregation in the blood sample indicates that the subject is afflicted with a disease or disorder selected from the group consisting of thrombosis, optionally an ischemic stroke; myocardial infarction; pulmonary embolism; deep vein thrombosis; and an infection, optionally a viral infection, optionally a viral infection caused by a positive-sense, single-stranded RNA virus, optionally a betacoronavirus, optionally SARS-CoV-2, SARS-CoV, or MERS-CoV

In some embodiments of this aspect, detecting aberrant red blood cell aggregation is performed by a method that comprises (a) separating mononuclear cells from non-aggregated red blood cells in the blood sample and (b) determining if, after separation, aggregated red blood cells are associated with the mononuclear cells.

In some embodiments of this aspect, separating mononuclear cells from non-aggregated red blood cells in a blood sample comprises performing a method selected from the group consisting of centrifugation, optionally density gradient centrifugation, sedimentation, and filtration.

In some embodiments of this aspect, determining if aggregated red blood cells are present in the sample comprises performing a method selected from the group consisting of visual inspection, spectroscopy, interferometry, electrochemistry, chromatography (optionally lateral flow immunochromatography, Raman scattering (SERS) (optionally surface-enhanced Raman scattering (SERS)), field-effect transistor (FET)-based biosensing, surface plasmon resonance (SPR)-based biosensing, a photoacoustic method, and an ultrasound method.

In some embodiments of this aspect, the presence of aberrant red blood cell aggregation in the blood sample indicates that the subject (i) has a disease or disorder characterized by aberrant red blood cell aggregation, optionally a viral infection or (ii) has not recovered from the a disease or disorder characterized by aberrant red blood cell aggregation, optionally a viral infection, wherein optionally the viral infection is caused by a positive-sense, single-stranded RNA virus, optionally a betacoronavirus, optionally SARS-CoV-2, SARS-CoV, or MERS-CoV, wherein the method optionally further comprises performing a second diagnostic method different from the method according to claim 1, wherein the second diagnostic method is optionally selected from the group consisting of diagnostic imaging method, a pathogen nucleic acid detection method (optionally a genome or ribosomal RNA detection method), an immunological method (optionally an immunoassay), a serological method, a molecular diagnostic assay, and the subject's clinical symptoms, and wherein the second diagnostic method is further optionally selected from the group consisting of a viral genome detection method; a detection method based on detecting an antibody response in the subject to the virus causing the viral infection; a detection method based on detecting a T cell response in the subject to the virus causing the viral infection; a blood clot formation assay, optionally a D-dimer assay; a myocardial infarction detection assay, optionally a BNP assay or a cardiac troponin assay; and a detection method based on presentation by the subject of one or more clinical symptoms indicative of infection by the virus causing the viral infection.

In some embodiments of this aspect, the methods are used to stratify the subject based on disease severity or stage, wherein optionally a degree of aberrant red blood cell aggregation is used to stratify the subject based on disease severity or stage.

In some embodiments of this aspect, the absence of aberrant red blood cell aggregation in a second blood sample, optionally a peripheral blood sample, obtained from the subject known to have been be afflicted with a disease or disorder characterized by aberrant red blood cell aggregation, indicates that the subject has recovered from the disease or disorder.

In a related aspect, the invention concerns methods for detecting an infection caused by a positive-sense, single-stranded RNA virus, optionally a betacoronavirus, optionally SARS-CoV-2, SARS-CoV, or MERS-CoV. Such methods involve using centrifugation, optionally density gradient centrifugation, to separate a mononuclear cells from non-aggregated red blood cells in a blood sample, optionally a peripheral blood sample, obtained from a human subject known or suspected to be infected with the virus, followed by determining if, after centrifugation, aggregated red blood cells are present between the separated mononuclear cells and non-aggregated red blood cells, which aggregated red blood cells, if present, indicates that the human subject is infected with the virus or has not recovered from infection by the virus.

In some embodiments of this aspect, when the method indicates that the subject is infected with the virus or has not recovered from infection by the virus, the method further involves combining that result with a result of another diagnostic method useful in diagnosing infection with the virus, wherein the other diagnostic method optionally is selected from the group consisting of a viral genome detection method, a detection method based on detecting an antibody response in the subject to the virus, a detection method based on detecting a T cell response in the subject to the virus, and a detection method based on presentation by the subject of one or more clinical symptoms indicative of infection by the virus.

In some embodiments of this aspect, the method involves determining if aggregated red blood cells are present in the sample comprises performing a method selected from the group consisting of visual inspection, spectroscopy, interferometry, electrochemistry, chromatography (optionally lateral flow immunochromatography, Raman scattering (SERS) (optionally surface-enhanced Raman scattering (SERS)), field-effect transistor (FET)-based biosensing, surface plasmon resonance (SPR)-based biosensing, a photoacoustic method, and an ultrasound method.

In some embodiments of this aspect, the presence of aberrant red blood cell aggregation in the blood sample indicates that the subject (i) has a viral infection or (ii) has not recovered from the viral infection, wherein the method optionally further comprises performing a second diagnostic method different from the method according to claim 10, wherein the second diagnostic method is optionally selected from the group consisting of diagnostic imaging method, a pathogen nucleic acid detection method (optionally a genome or ribosomal RNA detection method), an immunological method (optionally an immunoassay), a serological method, a molecular diagnostic assay, and the subject's clinical symptoms, and wherein the second diagnostic method is further optionally selected from the group consisting of a viral genome detection method; a detection method based on detecting an antibody response in the subject to the virus causing the viral infection; a detection method based on detecting a T cell response in the subject to the virus causing the viral infection; a blood clot formation assay, optionally a D-dimer assay; a myocardial infarction detection assay, optionally a BNP assay or a cardiac troponin assay; and a detection method based on presentation by the subject of one or more clinical symptoms indicative of infection by the virus causing the viral infection.

In some embodiments of this aspect, the methods are used to stratify the subject based on disease severity or stage, wherein optionally a degree of aberrant red blood cell aggregation is used to stratify the subject based on disease severity or stage.

In some embodiments of this aspect, the methods are used to determine if a human subject has recovered from a viral infection caused by a positive-sense, single-stranded RNA virus, optionally a betacoronavirus, optionally SARS-CoV-2, SARS-CoV, or MERS-CoV, comprising performing the method of claim 10 on a human subject known or suspected to have been infected by the virus and if no aggregated red bloods cells are detected, determining that the human subject has recovered from the viral infection.

These and other aspects and embodiments of the invention will be apparent to those of skill in the art upon reading the specification.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A-FIG. 1B. PBMC purification of samples from heathy individuals (FIG. 1A) or hospitalized individuals (FIG. 1B). Plasma was separated from the whole blood as described in the “Methods” section. Cell pellets were reconstituted with an equal volume of 1×PBS and then diluted 1:1 with 1×PBS prior to being over-laid on a density gradient medium.

FIG. 2A-FIG. 2C. Blood smears from the PBMC-medium interface of two samples following the centrifugation step in the PBMC purification process. (FIG. 2A, Healthy Control. FIGS. 2B & 2C, COVID-19+ patient sample.

FIG. 3A-FIG. 3C. Flow Cytometric Analysis of RBCs. Cells were gated on either CD41 a (Platelets) or CD235a (RBC). Two gates were used for the RBCs as shown by green (R5) and red (R4) arrows. Gated populations were further examined for Caspase 3/7 vs either CD95 (RBC) or CD178 (Platelets). (FIG. 3A) Whole Blood from a non-COVID sample. (FIG. 3B) Hospitalized patient that was RT-PCR COVID-19-negative but coded within five days of hospital admission. Cells examined were from the “Red Ring” formed at the PBMC-medium interface. (FIG. 3C) RBC pellet.

### DETAILED DESCRIPTION OF THE INVENTION

The object of this invention is address shortcomings in accurately and rapidly diagnosing diseases, disorders, or conditions characterized by aberrant red blood cell aggregation. Among these diseases and conditions are infections caused by RNA viruses, particularly those caused by positive-sense, single-stranded RNA viruses, known to cause human disease. Examples of such viruses include various betacoronaviruses, including SARS-CoV, MERS-CoV, and SARS-CoV-2. As is known, SARS-CoV-2 causes COVID-19, a potentially fatal illness.

In one aspect, the invention concerns methods for detecting an infection of a human subject by a positive-sense, single-stranded RNA virus, particularly a betacoronavirus, for example, SARS-CoV-2, SARS-CoV, or MERS-CoV. Broadly, such method involve determining whether aggregated red blood cells are present in a blood sample obtained from a subject, especially a human subject, known or suspected to be infected with the virus to be detected. Preferably, the blood sample is a peripheral blood sample, and if aggregated red blood cells are detected in the sample, such aggregation indicates that the subject is infected with the virus or has not recovered from infection by the virus.

In some embodiments, determining whether aggregated red blood cells are present in a subject's blood sample involves separating mononuclear cells from non-aggregated red blood cells in the blood sample and then determining if, after separation, aggregated red blood cells are associated with the mononuclear cells separated from the non-aggregated red blood cells. If so, the association indicates that the subject is infected with the virus. In some embodiments, the separation of mononuclear cells from non-aggregated red blood cells in a blood sample includes performing one or more of a centrifugation (for example, a density gradient centrifugation), sedimentation, or filtration procedure on the sample. In some embodiments, determining whether aggregated red blood cells are associated with the mononuclear cells separated from non-aggregated red blood cells in the sample involves performing a method such as visual inspection, spectroscopy, interferometry, electrochemistry, chromatography (optionally lateral flow immunochromatography, Raman scattering (SERS) (optionally surface-enhanced Raman scattering (SERS)), field-effect transistor (FET)-based biosensing, surface plasmon resonance (SPR)-based biosensing, a photoacoustic method, or an ultrasound method on the sample post-separation.

In certain embodiments, if the method indicates that the subject is infected with the virus or has not recovered from infection by the virus, that result is further combined with a result of another diagnostic method that can also be used to diagnose infection with the virus. Representative examples of such other methods include molecular diagnostic methods such as viral genome (or other viral nucleic acid, e.g., mRNA) detection (for example, via PCR or another nucleic acid amplification and/or detection technique), detection of an antibody response (e.g., IgG, IgM, antibodies) in the subject to the virus, detection of a T cell response in the subject to the virus, and detection of infection based on presentation by the subject of one or more clinical symptoms indicative of infection by the virus, such as fever or chills, cough, loss of taste and/or smell, shortness of breath or difficulty breathing, fatigue, muscle or body ache, headache, sore throat, congestion, nausea and/or vomiting, and/or diarrhea or other gastrointestinal upset. In some embodiments, examples of other molecular diagnostic assays include those useful in detecting blood clot formation (for example, a D-dimer assay), myocardial infarction (for example, a BNP assay and/or a cardiac troponin assay), etc.

In other embodiments, the method of the invention indicates that the subject is infected with the virus or has not recovered from infection by the virus. In addition, or alternatively, the method can be used to stratify the subject based on disease severity or stage.

In a related aspect, the invention is directed to methods for detecting an infection by a positive-sense, single-stranded RNA virus, for example, a betacoronavirus such as SARS-CoV-2, SARS-CoV, or MERS-CoV using centrifugation, preferably density gradient centrifugation, to separate a mononuclear cells from non-aggregated red blood cells in a blood sample (e.g., a peripheral blood sample) obtained from a human subject known or suspected to be infected with the virus, and after separating mononuclear cells from non-aggregated red blood cells by centrifugation, determining if aggregated red blood cells are associated with the separated mononuclear cells or layered between the mononuclear cells and non-aggregated red blood cells. If so, the aggregated red blood cells indicate that the human subject is infected with the virus or has not recovered from infection by the virus.

Another aspect of the invention concerns the invention involves detecting a condition, disease, or disorder associated with aberrant red blood cell aggregation independent of clot detection. Such methods typically comprise determining whether aggregated red blood cells are present in a blood sample (preferably a peripheral blood sample) obtained from a subject, preferably a human subject, known or suspected have the condition, disease, or disorder. If aggregated red blood cells are detected, such aggregation indicates that the subject has the condition, disease, or disorder.

In some embodiments of this aspect, the condition, disease, or disorder is associated with thrombosis, for example, an ischemic stroke, myocardial infarction, pulmonary embolism, or deep vein thrombosis. Alternatively, the condition, disease, or disorder is associated with a pathogenic or a viral infection, for example, an infection caused by a positive-sense, single-stranded RNA virus, for example, a betacoronavirus, examples of which include SARS-CoV-2, SARS-CoV, and MERS-CoV.

When the method detects that the subject has the condition, disease, or disorder, in some embodiments that result is then combined with the results of one or more other results obtained by performing at least another, preferably different diagnostic method useful to detect the condition, disease, or disorder. Of course, in some embodiments the results of one diagnostic method are confirmed by repeating that method and then comparing the results of each test to confirm whether the results are the same of different. As with other aspects of the invention, second or alternative diagnostic methods include diagnostic imaging methods, a pathogen nucleic acid detection methods (e.g., a genome or mRNA detection method), immunological methods (e.g., an immunoassay), a serological method, a molecular diagnostic assay, and patient symptoms.

Representative Methods

Blood Sample Acquisition and Preparation

Patient peripheral blood samples for clinical immunophenotype testing were obtained via a venipuncture into either an EDTA or heparin coated vacutainer tubes (BD Bioscience).

Plasma was removed from whole blood by centrifugation at 961 RFC for 5 minutes at room temperature (18-25° C.) and frozen in vapor phase liquid nitrogen. An equal volume of 1× phosphate buffered saline pH 7.2 (PBS) (Thermo Fisher Scientific, Carlsbad, Calif.) was added back to the blood cell pellet to effect resuspension. Peripheral blood mononuclear cells (PBMC) were separated from 2 mL of resuspended blood cells diluted 1:1 with PBS using 3 ml Lymphoprep (Stem cell Technologies, Cambridge, Mass.). The lymphoprep was added to either a 15 ml Falcon tube or Accuspin tube (Sigma-Aldrich, St. Louis, Mo.) per the manufacturer's directions. Resuspended blood cells were carefully layered onto the lymphoprep in 15 ml Falcon tubes or added to the Accuspin tubes as instructed. As a direct substitute for Lymphoprep/Accuspin, an Accuspin-Histopaque 1077 system (Sigma-Aldrich, St. Louis, Mo.) was also tested as per manufacturer's instructions.

After centrifugation, and independent of the particular system used (i.e., Falcon tube, Lymphoprep/Accuspin, or the Accuspin-Histopaque 1077 system), the PBMC layer from each tube was removed and washed in PBS and then resuspended in 0.5 mL PBS for additional processing.

Flow Cytometry

Whole Blood, PMBC/RBC layer obtained from the density gradient separation, or pelleted RBC from the density gradient separation were washed with 1×PBS. Samples were diluted 1:250 in HBSS. For each sample, 50 μl was then incubated with a Fam-FLICA probe specific for Caspase 3/7 (ImmunoChemistry Technologies, MN, Cat #93) for 1 hour at 37° C. Cells were washed with 1× Apoptosis buffer to remove unbound FLICA probes per manufacturer's directions. Washed cells were stained with CD41a (SB436), CD235a (APC), CD178 (PE), and CD95 (PE-Cy7) (Thermo Fisher Scientific, Carlsbad, Calif.) for 30 minutes then washed and run on a 3 laser BD FACS Canto 10.

CS&T beads (BD Bioscience, San Jose, Calif.) were acquired daily to ensure consistent performance of the Canto 10. The BD FACS Canto 10 was cleaned with 10 minutes of 10% bleach and water following acquisition of samples.

Results

Healthy and Covid-19+

Whole blood samples were initially fractionated to separate plasma from the RBCs and PBMCs for storage in liquid nitrogen. After removal of the plasma, an equal volume of 1×PBS was added back to the pelleted cells. Resuspended cells were further diluted 1:1 with 1×PBS prior to being overlayed onto 3 ml lymphoprep (or Histopaque). Where sample volume permitted, 2 ml of the cell-containing sample was diluted with 1×PBS 1:1 and over-laid onto the density gradient material. However, in some cases, a smaller sample volume was used. Several different PBMC purification methods were examined: 1) Lymphoprep/15 ml conical tube; 2) lymphoprep/Accuspin tubes with a porous frit; and 3) Accuspin-Histopaque 1077. The differences between these systems were primarily ease of use and process efficiency, where the Lymphoprep and 15 ml falcon tubes required slow and more precise layering of the blood samples. The Accuspin tubes incorporated a porous frit that allowed easier addition of sample, and the Accuspin-Histopaque 1077 system came ready to use. Despite these differences, similar results were obtained from each of these systems.

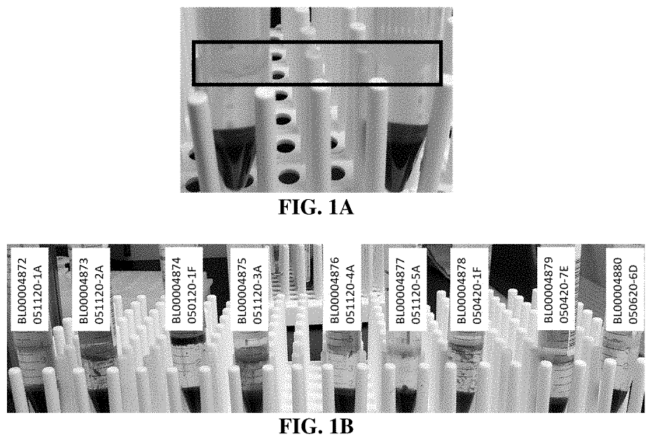

Post-centrifugation, red blood cell banding was examined. In healthy, non-COVID samples, the erythrocytes pelleted at the bottom of the tubes and upwardly displaced the density gradient. A PBMC band at the saline/plasma-medium interface without erythrocyte contamination as shown in FIG. 1A. In Covid-19+ samples, a similar banding pattern occurred but with a clear, distinct difference. Specifically, there was a contaminating layer of RBC/platelets below and adjacent to the PBMC band that could not be separated from the PBMC band. The RBC band varied in thickness between samples, as shown in FIG. 1B.

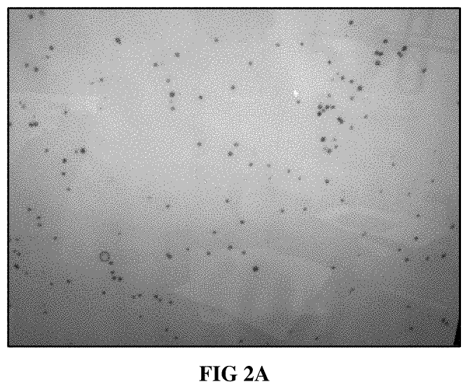

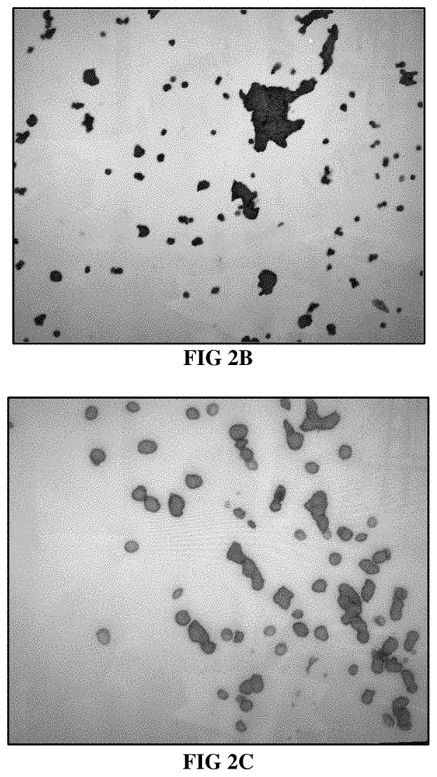

Blood smears were made from both non-COVID-19 (FIG. 2A) and COVID-19+(FIGS. 2B and 2C) samples, taken from the pelleted erythrocytes as well as from the PBMC/RBC co-band (“Red Ring”) interface and stained. As shown in FIGS. 2A and 2B, there was significant RBC aggregation or agglutination in the COVID-19+ samples. Of note, plasma had been removed and substituted with 1×PBS prior to the Lymphoprep processing step. The removal of plasma and continued RBC aggregation suggests the observed aggregation/agglutination is not rouleaux formation.

To determine if the PBMC/RBC co-band could be resolved into separate bands (or layers), a sample was washed and re-purified with Lymphoprep. The RBC layer continued to co-band with the PBMC layer (data not shown).

As shown in Table 1, below, all samples were from admitted ICU or non-ICU patients. Testing post-admittance ranged from 1 to 27 days. The Red Ring was observed in all samples, including samples that tested negative by RT-PCR for SARS-CoV-2.

TABLE 1
Description of samples shown in FIG. 1B. All samples were from hospitalized in-patients.

| | | | | | PBMC Purification |
|---|---|---|---|---|---|
| | | Date of | | | Days Post- |
| ID | Covid 19 Testing | Admittance | Outcome | Location | Admittance |
| 4872 | Negative (5/11) | May 11, 2020 | DC May 14, 2020 | IN-non icu | 1 |
| 4873 | Positive (5/10) | May 10, 2020 | DC May 14, 2020 | IN-non icu | 1 |
| 4874 | Negative (4/25, 4/27, 5/5, 5/14, | Apr. 26, 2020 | Expired | ICU | 15 |
| | 5/18) (igG Positive 5/18) | | May 23, 2020 | | |
| 4875 | Negative (4/20, 4/28) | Apr. 28, 2020 | DC May 12, 2020 | non icu | 13 |
| 4876 | Negative (5/12, 5/14) | May 12, 2020 | DC May 16, 2020 | IN-non icu | 1 |
| 4877 | Negative | May 11, 2020 | DC May 19, 2020 | IN-non icu | 1 |
| 4878 | Positive (4/21) | Apr. 21, 2020 | Expired | ICU | 20 |
| | | | May 17, 2020 | | |
| 4879 | Positive (Apr. 1, 2020) | Apr. 14, 2020 | DC May 20, 2020 | ICU | 27 |
| | Negative 5/19, 5/20 | | | | |
| 4880 | Positive 4/16, 5/11, 5/15 | Apr. 16, 2020 | Still Admitted | ICU | 25 |
| | | | on Jun. 8, 2020 | | |

Health Care Workers

Whole blood samples were obtained from health care workers (HCWs) and processed as previously described. RT-PCR testing results, flu-like symptoms since January 2020, or COVID-19-specific IgG results are shown in the Table 2, below, along with an image of the PBMC-medium interface from each sample. HCWs with a positive PCR test and positive IgG test all had a ‘Red Ring” at the PBMC-medium interface, including the HCW that did not have flu-like symptoms. Samples from the three of HCWs that were negative by PCR, IgG, and symptoms did not have a “Red Ring” at the PBMC-medium interface of their respective samples. As shown in Table 2, there multiple patterns were observed, but in all cases, except for HCW 5002, a “Red Ring” was present when COVID-19 IgG was detected. A “Red Ring” was also observed in one health care worker who did not experience flu-like symptoms or have a positive COVID-19 IgG result. With one exception (HCW 5070), a ‘Red Ring” was observed in all HCWs who experienced flu-like symptoms. Of note, the “Red Ring” banding was smaller or less pronounced in HCWs than in patients admitted to the hospital and who were tested within a month of admission, indicating that “Red Ring” banding becomes smaller or less pronounced with increased time post-infection.

In Table 2, samples were grouped initially by PCR test result or lack thereof. They were then further sorted by symptoms and IgG testing results. A corresponding image of the PMBC-medium interface from the PBMC purification is shown in the far-right column of the Table.

FasR, FasL, Caspase 3/7 in RBC

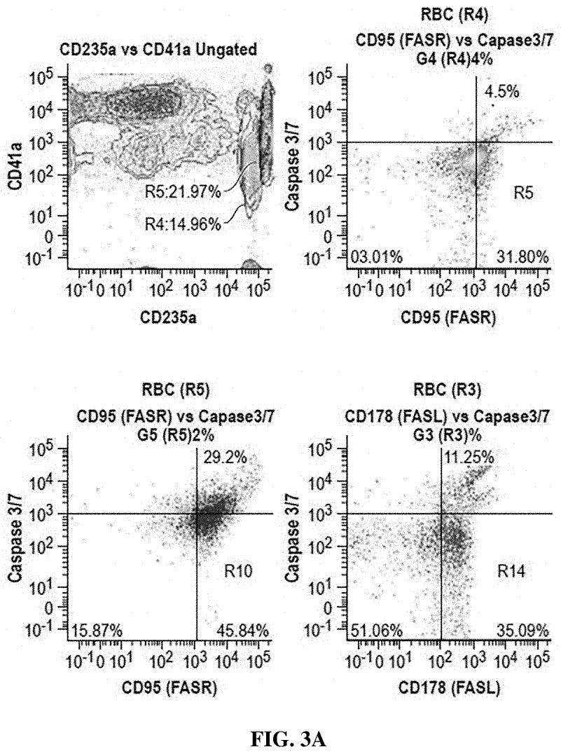

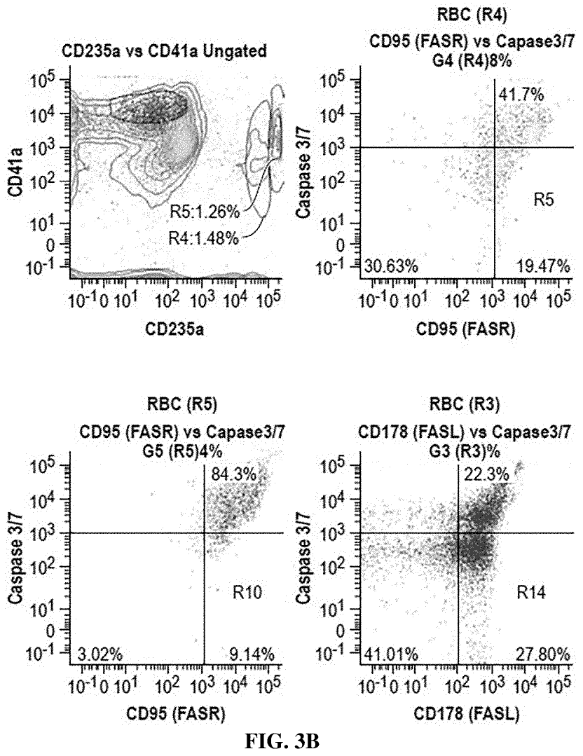

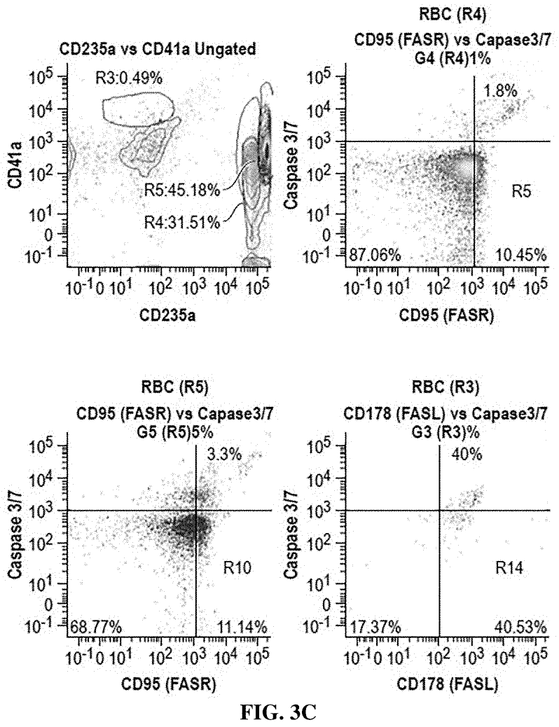

RBC and platelets were examined for the expression of both CD95 & Caspase 3/7 or CD178 and Caspase 3/7, respectively. As shown in FIG. 3, there was a clear increase in the percent of CD95 (FasR)+Caspase 3/7+ cells in the hospitalized patient in both the R4 and R5 gated RBC populations (FIG. 3B) over the healthy patient (FIG. 3A). When comparing RBCs in the “Red Ring” RBC to those in pelleted RBCs, there was also a clear increase in the percent CD95 (FasR)+Caspase 3/7+ cells in the “Red Ring” population. This result indicates that the RBCs in the “Red Ring” are undergoing Fas-mediated cell death and aggregation. Similarly, there was an increase in the percent of CD178+(FasL) Caspase 3/7+ platelets in the “Red Ring” as compared to the healthy control.

Conclusions:

The “Red Ring” structure was observed in all hospitalized patient samples tested with variance in size and intensity, and it was not observed in healthy controls. In several cases where a “Red Ring” was observed, RT-PCR testing did not indicate COVID-19 positivity. In several patients that had initial positive RT-PCR test result followed by subsequent negative PCR results, the “Red Ring” was still observed in patient samples. In hospitalized patients, the “Red Ring” was observed as early as one day post-admittance to over 27 days post-admittance, indicating that screening for a “Red Ring” (or an associated or corresponding feature detectable using a different testing platform) is a quick test to indicate COVID-19 status.

When examined in health care workers, the “Red Ring” was not observed in all individuals. However, time post-symptom and potential exposure was unknown. Despite the unknown timing for symptoms and exposure, there was a correlation between symptoms, COVID-19 IgG test results, and “Red Ring” formation during PBMC purification. Together, these results indicate that the presence or absence of a “Red Ring” (or an associated or corresponding feature detectable using a different testing platform) is not only an indication of COVID-19 infection status, but of a subsequent recovery from COVID-19 infection.

In addition to COVID-19 detection, the invention also has application with regard in the context of detecting. As is known, thrombotic events are among the many complications of COVID-19 [2][3]. Given the significantly altered physiological state of patients with increased inflammatory markers and oxidative stress, platelets and RBCs are stressed. Mechanistically, in COVID-19 patients, activated platelets expose FasL on their exterior surface, allowing subsequently binding to FasR on RBCs. This FasL-FasR interaction triggers the alteration in phosphatidylserine (PS) exposure on the outer surface of the RBC cell membrane, consequently leading to eryptosis [4][5] [6]. While not being bound to any particular theory, this process can lead to the aggregation of RBCs observed in the “Red Ring” structures seen after density gradient separation of blood samples from known or suspected COVID-19 patients. The degree or size (or other measure) of a “Red Ring” observed in a sample can also be an indication of potential thrombotic event likelihood or severity.

### REFERENCES

1. AAFP. COVID 19 Testing—Guide for Physicians. 2020 Jul. 10, 2020; Available from: https://www.aafp.org/patient-care/emergency/2019-coronavirus/covid-19_resources/covid-19--testing.html.
2. Ferner, R. E., Levi, M., Sofat, R., Aronson, J. K. Thrombosis in COVID-19: clinical outcomes, biochemical and pathological changes, and treatments. 2020; Available from: https://www.cebm.net/covid-19/thrombosis-in-covid-19-clinical-outcomes-biochemical-and-pathological-changes-and-treatments/.
3. Connors, J. M. and J. H. Levy, COVID-19 and its implications for thrombosis and anticoagulation. Blood, 2020. 135(23): p. 2033-2040.
4. Boulet, C., C. D. Doerig, and T. G. Carvalho, Manipulating Eryptosis of Human Red Blood Cells: A Novel Antimalarial Strategy? Frontiers in cellular and infection microbiology, 2018. 8: p. 419-419.
5. Klatt, C., et al., Platelet-RBC interaction mediated by FasL/FasR induces procoagulant activity important for thrombosis. J Clin Invest, 2018. 128(9): p. 3906-3925.
6. Schleicher, R., et al., Platelets induce apoptosis via membrane-bound FasL. Blood, 2015. 126(12): p. 1483-1493.

What is claimed:

1. A method for detecting aberrant red blood cell aggregation, comprising determining whether a blood sample, optionally a peripheral blood sample, obtained from a subject, optionally a human subject, known or suspected to be afflicted with a disease or disorder characterized by aberrant red blood cell aggregation, contains pathologic red blood cell aggregation, and if so, indicating that aberrant red blood cell aggregation has been detected in the sample.

2. A method according to claim 1 wherein disease or disorder characterized by aberrant red blood cell aggregation is selected from the group consisting of thrombosis, optionally an ischemic stroke; myocardial infarction; pulmonary embolism; deep vein thrombosis; and an infection, optionally a viral infection, optionally a viral infection caused by a positive-sense, single-stranded RNA virus, optionally a betacoronavirus, optionally SARS-CoV-2, SARS-CoV, or MERS-CoV

3. A method according to claim 1 wherein a presence of aberrant red blood cell aggregation in the blood sample indicates that the subject is afflicted with a disease or disorder selected from the group consisting of thrombosis, optionally an ischemic stroke; myocardial infarction; pulmonary embolism; deep vein thrombosis; and an infection, optionally a viral infection, optionally a viral infection caused by a positive-sense, single-stranded RNA virus, optionally a betacoronavirus, optionally SARS-CoV-2, SARS-CoV, or MERS-CoV

4. A method according to claim 1 wherein detecting aberrant red blood cell aggregation is performed by a method that comprises (a) separating mononuclear cells from non-aggregated red blood cells in the blood sample and (b) determining if, after separation, aggregated red blood cells are associated with the mononuclear cells.

5. A method according to claim 4 wherein separating mononuclear cells from non-aggregated red blood cells in a blood sample comprises performing a method selected from the group consisting of centrifugation, optionally density gradient centrifugation, sedimentation, and filtration.

6. A method according to claim 4 wherein determining if aggregated red blood cells are present in the sample comprises performing a method selected from the group consisting of visual inspection, spectroscopy, interferometry, electrochemistry, chromatography (optionally lateral flow immunochromatography, Raman scattering (SERS) (optionally surface-enhanced Raman scattering (SERS)), field-effect transistor (FET)-based biosensing, surface plasmon resonance (SPR)-based biosensing, a photoacoustic method, and an ultrasound method.

7. A method according to claim 1 wherein the presence of aberrant red blood cell aggregation in the blood sample indicates that the subject (i) has a disease or disorder characterized by aberrant red blood cell aggregation, optionally a viral infection or (ii) has not recovered from the a disease or disorder characterized by aberrant red blood cell aggregation, optionally a viral infection, wherein optionally the viral infection is caused by a positive-sense, single-stranded RNA virus, optionally a betacoronavirus, optionally SARS-CoV-2, SARS-CoV, or MERS-CoV, wherein the method optionally further comprises performing a second diagnostic method different from the method according to claim 1, wherein the second diagnostic method is optionally selected from the group consisting of diagnostic imaging method, a pathogen nucleic acid detection method (optionally a genome or ribosomal RNA detection method), an immunological method (optionally an immunoassay), a serological method, a molecular diagnostic assay, and the subject's clinical symptoms, and wherein the second diagnostic method is further optionally selected from the group consisting of a viral genome detection method; a detection method based on detecting an antibody response in the subject to the virus causing the viral infection; a detection method based on detecting a T cell response in the subject to the virus causing the viral infection; a blood clot formation assay, optionally a D-dimer assay; a myocardial infarction detection assay, optionally a BNP assay or a cardiac troponin assay; and a detection method based on presentation by the subject of one or more clinical symptoms indicative of infection by the virus causing the viral infection.

8. A method according to claim 7 used to stratify the subject based on disease severity or stage, wherein optionally a degree of aberrant red blood cell aggregation is used to stratify the subject based on disease severity or stage.

9. A method according to claim 1 wherein the absence of aberrant red blood cell aggregation in a second blood sample, optionally a peripheral blood sample, obtained from the subject known to have been be afflicted with a disease or disorder characterized by aberrant red blood cell aggregation, indicates that the subject has recovered from the disease or disorder.

10. A method for detecting an infection caused by a positive-sense, single-stranded RNA virus, optionally a betacoronavirus, optionally SARS-CoV-2, SARS-CoV, or MERS-CoV, comprising (a) using centrifugation, optionally density gradient centrifugation, to separate a mononuclear cells from non-aggregated red blood cells in a blood sample, optionally a peripheral blood sample, obtained from a human subject known or suspected to be infected with the virus and (b) determining if, after centrifugation, aggregated red blood cells are present between the separated mononuclear cells and non-aggregated red blood cells, which aggregated red blood cells, if present, indicates that the human subject is infected with the virus or has not recovered from infection by the virus.

11. A method according to claim 10 that, when the method indicates that the subject is infected with the virus or has not recovered from infection by the virus, further comprises combining that result with a result of another diagnostic method useful in diagnosing infection with the virus, wherein the other diagnostic method optionally is selected from the group consisting of a viral genome detection method, a detection method based on detecting an antibody response in the subject to the virus, a detection method based on detecting a T cell response in the subject to the virus, and a detection method based on presentation by the subject of one or more clinical symptoms indicative of infection by the virus.

12. A method according to claim 10 wherein determining if aggregated red blood cells are present in the sample comprises performing a method selected from the group consisting of visual inspection, spectroscopy, interferometry, electrochemistry, chromatography (optionally lateral flow immunochromatography, Raman scattering (SERS) (optionally surface-enhanced Raman scattering (SERS)), field-effect transistor (FET)-based biosensing, surface plasmon resonance (SPR)-based biosensing, a photoacoustic method, and an ultrasound method.

13. A method according to claim 10 wherein the presence of aberrant red blood cell aggregation in the blood sample indicates that the subject (i) has a viral infection or (ii) has not recovered from the viral infection, wherein the method optionally further comprises performing a second diagnostic method different from the method according to claim 10, wherein the second diagnostic method is optionally selected from the group consisting of diagnostic imaging method, a pathogen nucleic acid detection method (optionally a genome or ribosomal RNA detection method), an immunological method (optionally an immunoassay), a serological method, a molecular diagnostic assay, and the subject's clinical symptoms, and wherein the second diagnostic method is further optionally selected from the group consisting of a viral genome detection method; a detection method based on detecting an antibody response in the subject to the virus causing the viral infection; a detection method based on detecting a T cell response in the subject to the virus causing the viral infection; a blood clot formation assay, optionally a D-dimer assay; a myocardial infarction detection assay, optionally a BNP assay or a cardiac troponin assay; and a detection method based on presentation by the subject of one or more clinical symptoms indicative of infection by the virus causing the viral infection.

14. A method according to claim 10 used to stratify the subject based on disease severity or stage, wherein optionally a degree of aberrant red blood cell aggregation is used to stratify the subject based on disease severity or stage.

15. A method according to claim 10 for determining if a human subject has recovered from a viral infection caused by a positive-sense, single-stranded RNA virus, optionally a betacoronavirus, optionally SARS-CoV-2, SARS-CoV, or MERS-CoV, comprising performing the method of claim 10 on a human subject known or suspected to have been infected by the virus and if no aggregated red bloods cells are detected, determining that the human subject has recovered from the viral infection.
